(19) United States
(12) Patent Application Publication
(10) Pub. No.: US 2017/0238935 A1
(43) Pub. Date: Aug. 24, 2017

(54) Duplex Tissue Clamp and Applier

(71) Applicant: Qingqing SHI, Nantong (CN)

(72) Inventor: Qingqing SHI, Nantong (CN)

(21) Appl. No.: 15/519,558

(22) PCT Filed: Jul. 6, 2016
(86) PCT No.: PCT/CN2016/088862

§ 371 Date: Apr. 17, 2017

(30) Foreign Application Priority Data
Aug. 26, 2015 (CN) 2015105318458

(51) Int. Cl. A61B 17/122; A61B 17/128

(52) U.S. Cl. CPC A61B 17/122; A61B 17/1285

(57) ABSTRACT

Disclosed is a duplex tissue clamp, which is provided with two tissue clamps of a parallel structure. Each of the tissue clamps comprises a V-shaped clamp body integrally formed by a first arc-shaped clamp arm (5) and a second arc-shaped clamp arm (6). The free ends of each first arc-shaped clamp arm (5) and the corresponding second arc-shaped clamp arm (6) are respectively a clamping hook (4) and a clamping groove (7, 7-1). A “hinge” structure is formed between each first arc-shaped clamp arm (5) and the corresponding second arc-shaped clamp arm (6). Each V-shaped clamp body is formed when the corresponding first arc-shaped clamp arm (5) and the corresponding second arc-shaped clamp arm (6) are in a free state. The two tissue clamps are provided with the two clamping grooves (7, 7-1) corresponding to the clamping hooks (4).

### TECHNICAL FIELD

This invention involves an auxiliary medical instrument for surgical operations, particularly a clamp used for sealing blood vessels or tissues, also known as the vascular clip or ligating clip.

### BACKGROUND TECHNOLOGY

During endoscopic surgery, it is necessary to seal broken blood vessels in the surgical incision to stop bleeding. However, the practice of using hemostatic forceps to nip a broken blood vessel and then finishing ligation with seams does not conform to the requirements of being quick and easy for endoscopic surgery. Hemostatic tissue clamps made of polymeric materials have been widely applied. A tissue clamp includes a V-shaped clamping body consisting of the first curved clamping arm and the second curved clamping arm. The free ends of the first and second curved clamping arms are a clamping hook and a clamping groove, respectively; and the other ends of the clamping arms are connected with a ‘hinge’ structure, which is described as follows: the joint between the first and second curved clamping arms comprise an internal connector and an external connector, arranged in a furcated manner. There is a hole between the connectors. When the first and second curved clamping arms form a V-shaped clamping body, the hole appears U-shaped. When the clamping hook and groove at the free ends of the first and second curved clamping arms are engaged with each other, the tissue clamp is closed to form a narrow gap which stops bleeding. The stress resulting from the arc-shaped structure of the first and second curved clamping arms makes the hook and groove engage tightly. Whereas the pressure exerted on a biological tissue may not cause it to die or to operate, usually there is a narrow gap left between the clamping arms closed. And the clamping surfaces of the arms feature several tooth-shaped zones, which help generate a relatively larger friction between the biological tissue and the clamping arms.

Tissue clamps usually have three to five size specifications, with series-specific clamp appliers and releasers, to use for tissues of different sizes. The appliers and releasers are equipped with a long handle to facilitate use in endoscopic surgery. Of course, tissue clamps can also be made of metals or absorbable materials.

The above design schemes are based on existing techniques, which have been known to the public and widely used by hospitals as surgical kits. Typical Chinese patents and patent applications include: CN201110023843, 201310334138 and 201410777554. However, the existing techniques are still deficient: although the reliability of existing clamps (with a gap on each clamp) is quite good, it is still generally necessary for surgeons to apply a second clamp in practice, and sometimes such second clamp can hardly be applied at a remnant end in tissue incision. When it is required to grip completely or achieve a higher reliability, surgeons often apply a second clamp. The front end (with metallic jaws to clamp the bumps for force applying) of the clamp applier currently used in endoscopic surgery for operating the existing tissue clamp wraps the tissue clamp completely (as the tissue clamp is only 1 mm or less in width). Although an endoscope (or a special image-capturing device) in use can offer an observation window, the visual operative field will still be affected to the extent that sometimes it completely relies on hand feeling to operate a clamp applier to block a blood vessel. US2005/0171560A1 has disclosed the schematic diagrams of the clamp applier.

Many clamps need to be left in the body of a surgical patient for a relatively long time. In the case of clamping arteries or blood vessels in key parts of the body, doctors will choose to use two clamps for the sake of security as long as conditions permit doing so. However, in the case that the remnant end of a blood vessel turns out to be too short, there is no existing product and technique capable of applying two separate clamps simultaneously.

All tissue clamps based on existing techniques are designed as a single/individual clamp, with bumps located on the sides as the supporting points for a clamp applier. The bumps and outer rectangular angles of such a clamp may easily cause friction and injury to the tissue around the blood vessel, especially for patients with lung lobe surgery, who may consequently suffer cough and bleeding symptoms. The clamping arm heads of an ordinary tissue clamp consist of a clamping hook and a clamping groove. The clamping groove is formed by the bulges on both sides of the clamping arm (the second curved clamping arm). The profile of the middle section of the clamping groove's bottom is a wedge fitting the clamping hook. The clamping groove and hook can only work in the condition of locking/occluding rather than the double insurance of ‘locking’ and ‘clasping.’ For use with big blood vessels or blood vessels with remnant tissue, such a tissue clamp is easy to burst open, thus causing concerns about loose clamping. If tissue (such as a blood vessel) is relatively thick, such tissue clamp will be unable to hold it or hold it tight (as shown in FIG. 9).

The cross-section shape of the two clamping arms is rectangular (with edges only slightly polished) but it cannot be changed into an oval shape because an ordinary single clamp with an oval cross-section shape for its arms would be easy to produce torsion and get distorted in the course of clamping as a result of external forces (vertical clamping force or horizontal resistance force in blood vessel stripping).

The bulges on both sides of the clamping groove adopt the water-droplet shape, which can help guiding the clamping. However, when it comes to remove the clamp, if the operation is improper or the blood vessel is too thick or not adequately free of tissue, the single clamp will be distorted easily; and the top end is not rigid enough due to the water-droplet design and thus unable to guide the move effectively, so the clamp may be broken.

Usually, two clamps will be used for clamping one end of a blood vessel, and a small-sized clamp is 2 mm wide (clamping arm+bump) while a medium-/large-sized clamp is 3 mm wide. In practice, two clamps cannot be applied very closely so as to prevent sideslip caused by mutual extrusion. Furthermore, it is also impossible to place two clamps side by side closely as a result of restrictions of surgical instruments, doctor techniques and single clamp's bumps. So, theoretically, use of two single clamps requires a length of at least 2 mm+2 mm+1 mm (interval)=5 mm, or for medium-/large-sized clamps at least 3 mm+3 mm+1 mm (interval)=7 mm.

The traditional way of using two tissue clamps simultaneously to realize the effect of double insurance is actually not safe because if the artery bursts through the first clamp, the second clamp is right identical with that first one in terms of clamping force and anti-skid performance, thus unsafe. In practice, if only one clamp has been used for clamping a blood vessel, it is usually not because using one clamp is adequate but mostly because the remnant end of the blood vessel is too short, or the space for clamping is too narrow, or the exposure of free blood vessels is not fine enough in urgent surgery.

Moreover, the existing clamp applier wraps a tissue clamp for applying (i.e. the clamp is closed) and the vision is not clear enough to directly check whether the clamping of a blood vessel is well done. Especially for a blood vessel which is very thick or not free enough of tissue, chances are that the phenomenon of ‘fake clamping’ will occur, thus causing fatal collapse and slippage during or after surgery. In particular, the postoperative collapse is extremely easy to cause death or secondary surgery due to infection, and this belongs to medical malpractice. In addition, the clamping hook and clamping groove of a tissue clamp based on existing techniques can be further improved. The clamping groove is formed by the bulges on both sides of the clamping arm (the second curved clamping arm). The profile of the middle section of the clamping groove's bottom is a wedge fitting the clamping hook. For use with a big blood vessel or a blood vessel not free enough of tissue, such a tissue clamp is easy to burst open, which can hardly be detected, thus causing concerns about loose clamping.

### SUMMARY OF THE INVENTION

The purpose of this invention is to provide a duplex or double-head tissue clamp for overcoming or avoiding the aforesaid issues. The duplex tissue clamp features an integrated duplex or double-head structure and functions better than two separate clamps used together. Its width will not influence the use of an applier so it can meet the requirements for endoscope application (mainly meeting the requirement of diameter). The clamp can grip completely with higher reliability. The application of one double-head tissue clamp is equivalent to the use of two separate single tissue clamps, which can often be seen in surgery. In most cases, applying two single clamps simultaneously is beneficial and harmless.

In order to solve existing technical problems, this invention sets forth a technical scheme which adopts two single tissue clamps connected in a parallel manner. Each of the said paralleled tissue clamps includes a V-shaped clamping body formed by the first curved clamping arm and the second curved clamping arm. The free ends of the said first and second curved clamping arms are a clamping hook and a clamping groove, respectively. Between the first and second curved clamping arms there is a ‘hinge’ structure. The clamping surfaces of the clamping arms contain several ‘splayed’ anti-skid tooth zones. In their free status, the first and second curved clamping arms form the V-shaped clamping body. The first bump is located near the hooks on the adjacent inner sides of the first curved clamping arms, and it connects the adjacent sides to act as the third connecting rod tying the two paralleled tissue clamps together; the two paralleled tissue clamps have two clamping grooves corresponding to the hooks (usually the clamping grooves are formed by the upward bulges on the sides of the second curved clamping arm); there are upward bulges on the adjacent inner sides between the two clamping grooves, and the bulges are connected with each other to form the fourth connecting rod tying the two paralleled tissue clamps together; and such third and fourth connecting rods act as the supporting points for the clamp applier. A clamping groove can also be called a ‘clamping bulge,’ which clasps the neck of the corresponding clamping hook.

The fourth connecting rod is the upward bulge in the middle formed by merging two original upward bulges on the adjacent sides between the two clamping grooves, i.e. adopting only one upward bulge to guide the two clamping hooks simultaneously.

The first and/or second (one or two) connecting rods are located near the hinge and on the adjacent inner sides of the first curved clamping arms or the second curved clamping arms.

When the two paralleled tissue clamps are designed with an interval greater than 1.5-3 mm, the third and fourth connecting rods will also provide force-applying points for the clamp applier. When the two paralleled tissue clamps are designed with an interval greater than 1.5-3 mm, the middle upward bulge between the clamps will be wider; and there is a supporting groove in the middle upward bulge for supporting the clamp applier.

The clamping arms (i.e. clamp legs) of a medium-/large-sized duplex tissue clamp are 2*1 mm in width together, plus the interval of 2 mm of the third and fourth connecting rods (with a bolder cross-section), so the total width is 4 mm.

The first to fourth connecting rods of the two paralleled tissue clamps constitute a bump on the outer sides of the two paralleled tissue clamps, providing a force-applying point for the clamp applier; and the third and fourth connecting rods can extend in a linear direction from the outer sides of the curved clamping arms to form the second bump, functioning as another force-applying point for the clamp applier.

The clamping surfaces of the clamping arms are designed with several splay-styled anti-skid tooth zones (8). The teeth in the anti-skid tooth-shaped zones of each single tissue clamp look like oblique lines. If the two paralleled single tissue clamps are closed, the anti-skid tooth-shaped zones look like splayed lines; each line of oblique teeth is divided into an upper segment and a lower segment, of which the oblique teeth are arranged in a straight or staggered manner.

The clamping hook and clamping groove can fit into each other to form a double-fish-shaped hook, suggesting a Tai Chi Yin Yang Fish diagram. The hook and groove also act as the groove and hook, respectively, for each other; the groove is in a fish shape and the hook looks like a curve-tailed fish.

When the fourth connecting rod is very short, the two inner upward bulges between the two clamping grooves can merge into one middle upward bulge, i.e. adopting only one upward bulge to guide the two clamping hooks simultaneously. When the two paralleled tissue clamps are designed with an interval greater than 1.5-3 mm, the fourth connecting rod will be wider and its cross-section will also increase. A supporting groove will be designed in the fourth connecting rod for supporting the clamp applier.

Furthermore, the shape of this invention looks like a Tai Chi Double-Fish diagram after the two arms engage with each other (the clamping hook and clamping groove also work as the clamping groove and clamping hook, respectively, for each other). And the clamping hook section is fish-shaped. However, the shape of the connecting arc is identical or similar to that of the existing design.

Beneficial effect: the existing clamp applier used in endoscopic surgery wraps the tissue clamp completely; although in practice there is an endoscope (or a special image-capturing device) offering an observation window, the visual operative field will still be affected to the extent that sometimes it completely relies on hand feeling to operate a clamp applier to block a blood vessel. A duplex or double-head tissue clamp is hereby set forth for overcoming or avoiding the aforesaid issues. The duplex tissue clamp features an integrated duplex or double-head structure and functions better than two separate clamps used together. Its width will not influence the use of an applier so it can meet the requirements for endoscope application (mainly meeting the requirement of diameter). The clamp can grip completely with higher reliability. The application of one duplex or double-head tissue clamp is equivalent to the use of two separate single tissue clamps, which can often be seen in surgery.

Through the design of the duplex tissue clamp (i.e. the double-head tissue clamp), the bumps can be located on the inner sides rather than outer sides; and the connecting rods between the paralleled clamps can act as the bumps and provide the supporting points for the applier. The design moves the bumps from outer sides to inner sides, thus avoiding damaging blood vessels easily. Thanks to the ‘double-head/double-arm’ design, plus the three or four connecting rods on the two arms, the double-head tissue clamp is improved from two supporting points to four supporting points, thus enhancing the distortion resisting ability and making it possible to change the outer cross-section of the two arms from a square into an oval shape.

The connecting rods between the two arms can adopt a larger size to stabilize the structure, so that the two arms after engaged can be restricted by the connecting rods between them, avoiding the occurrence of lateral sway and deformation.

Through the design of the double-head tissue clamp, the bumps on the outer sides of the clamp can be reduced and meanwhile the spacing between the two paralleled clamps is fixed, so the width of a medium-/large-size double-head tissue clamp is 1 mm*2 (clamp legs)+2 mm (bold connecting rod)=4 mm, which is 3 mm smaller than that in the traditional method. Therefore, this design can save a lot of space in the application of vascular clamps and boast a great improvement in terms of vascular ligation techniques in laparoscopic surgery! And the length of a blood vessel for ligation required by a small-sized double-head tissue clamp is also shortened by 1.5 mm from 3.5 mm originally required.

The ‘double-head tissue clamp’ is not just a simple addition of 1+1. Compared with two separate tissue clamps used together, the double-head tissue clamp features a stable structure and provides a doubled clamping force for resisting the shock of blood flow, as the shock of blood flow can break through two traditional tissue clamps one by one with the same force each time. In addition, the gap between the two arms of a double-head tissue clamp leaves an adequate space for maintaining vascular activity. Meanwhile, as the blood vessel is extruded in the course of clamping, the vascular segment swells up between the two arms, thus achieving a very good anti-skid effect. Therefore, the double-head tissue clamp provides a clamping force twice as large as that offered by two separate traditional tissue clamps used together, and realizes an anti-sideslip resistance of 1+1>2, plus an extra resistance of >3 provided by the vascular segment between the two clamping arms.

And it is also easy to design a new clamp applier. The positions for clamp applying and releasing by the new clamp applier are located between the inner sides of the two arms, i.e. the connecting rods 2 and 2-1 (including the bumps/bulges in the positions); meanwhile, the T-shaped design is adopted to guarantee good stability and clear vision in the course of clamp operation, enabling a clear observation of results of clamp operation.

The improvements realized by this invention also include the design of the double-head tissue clamp's Tai Chi Double-Fish shaped hooks and grooves, which can not only avoid tissue injury caused by sharp clamp ends but also enable smoother operation than using sharp-ended traditional tissue clamps, thus avoiding the occurrence of abnormal cases, such as distortion and fracture due to unsmooth operation and resistance caused by the sharp ends; and meanwhile, the Tai Chi Double-Fish shaped design not only allows the engaging of the two ends of a clamp but also realizes the effect of clasping due to its structure of small inside and big outside, so that the probability of collapse of clamping becomes even lower. (Just like bending your fingers and making your hands clasp each other, with one hand facing yourself and the other hand facing the other way)

The beneficial effect of this invention: providing a duplex or double-head tissue clamp to overcome the deficiencies of existing tissue clamps. The duplex tissue clamp features an integrated duplex or double-head structure and functions as but better than two separate clamps used side by side. Its width design will not influence the use of an applier so it can meet the requirements for endoscope application (mainly meeting the requirement of diameter). The clamp can grip completely with higher reliability. The application of one double-head tissue clamp is equivalent to the use of two separate traditional tissue clamps simultaneously, which can often be seen in surgery. In most cases, applying two single clamps simultaneously is helpful. Due to its Tai Chi Double-Fish shaped design, the tissue clamp's clamping body is more reasonable. The reliability of engaging and locking is improved, and the injury to tissue caused by sharp ends (the outer edges and bumps of the clamping arms) is avoided.

### BRIEF DESCRIPTION OF THE DRAWINGS

FIG. 1 is a graphic model showing the invented duplex or double-head tissue clamp without any bump.

FIG. 2 is a graphic model showing the invented duplex or double-head tissue clamp with a bump structure.

FIG. 3 is a graphic model showing the invented duplex or double-head tissue clamp with another kind of bump structure.

FIG. 4 is a graphic model showing the invented duplex or double-head tissue clamp without any bump (with only one upward bulge in the middle).

FIG. 5 is a schematic diagram showing the invented duplex or double-head tissue clamp with a Tai Chi Double-Fish shaped hook-groove structure.

FIG. 6 is a schematic diagram showing the invented duplex or double-head tissue clamp with another kind of Tai Chi Double-Fish shaped hook-groove structure.

FIG. 7 is a schematic diagram showing the section structure of the clamping hooks and grooves of the invented duplex or double-head tissue clamp (with only one middle upward bulge), which is about to be closed.

FIG. 8 is a schematic diagram showing the section structure of the clamping hooks and grooves of the invented duplex or double-head tissue clamp (with two middle upward bulges), which is about to be closed.

FIG. 9 is a schematic diagram showing the section structure of the clamping hooks and grooves of the invented duplex or double-head tissue clamp (with two middle upward bulges with a processed profile), which is about to be closed.

FIG. 10 is a schematic diagram showing the section structure of the clamping arm and tooth.

FIGS. 11a and 11b are schematic diagrams showing the layout of the tooth zones on the opened clamping arms of the invented duplex or double-head tissue clamp; i.e. schematic diagrams showing the layout of the two types of teeth on the clamping arms.

FIG. 12 is a schematic diagram showing the section structure of engaged hooks and grooves of this invention, including a Tai Chi Double-Fish shaped hook-groove structure (FIG. 12a) and a variant structure (FIG. 12b).

FIG. 13 is a schematic diagram showing the section structure of the Tai Chi Double-Fish shaped hooks and grooves of the invented duplex or double-head tissue clamp, which is about to be closed.

FIG. 14 is a graphic model showing the existing single tissue clamp with a bulge structure.

FIG. 15 is a schematic diagram showing the Tai Chi Double-Fish shaped hook-groove structure of a single tissue clamp set forth by this invention.

FIG. 16 is a schematic diagram showing the Tai Chi Double-Fish shaped hook-groove structure of another single tissue clamp set forth by this invention.

FIG. 17 is a schematic diagram showing the Tai Chi Double-Fish shaped hook-groove structure of another single tissue clamp set forth by this invention.

FIG. 18 is a schematic diagram showing the Tai Chi Double-Fish shaped hook-groove structure of the invented clamp after closed (corresponding to FIG. 17).

FIG. 19 is a schematic diagram showing the Tai Chi Double-Fish shaped hook-groove structure of the invented clamp after closed (corresponding to FIG. 16).

FIG. 20 is a schematic diagram showing the Tai Chi Double-Fish shaped hook-groove structure of the invented clamp after closed (corresponding to FIG. 18).

FIG. 21 is a schematic diagram showing the Tai Chi Double-Fish shaped hook-groove structure of the invented clamp after closed (corresponding to FIG. 18).

FIG. 22 shows the shape of the engaged hook and groove of this invented clamp.

FIG. 23 is a schematic diagram showing the head part of the existing clamp applier.

FIG. 24 is a schematic diagram showing the clamping head of the clamp applier specially designed for operating the invented duplex or double-head tissue clamp.

### DETAIL DESCRIPTION

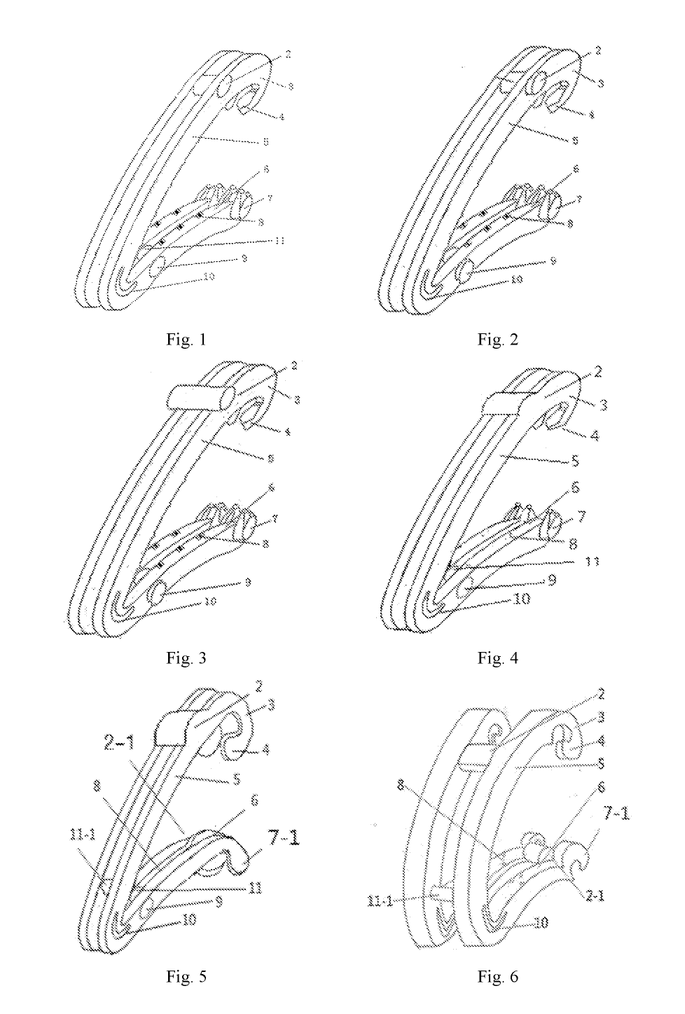

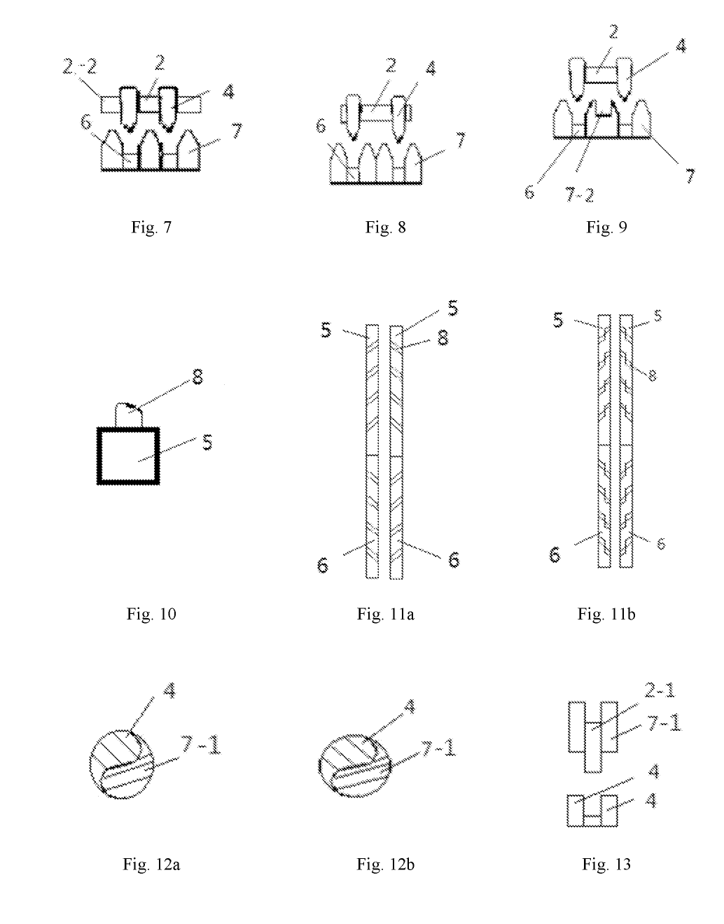

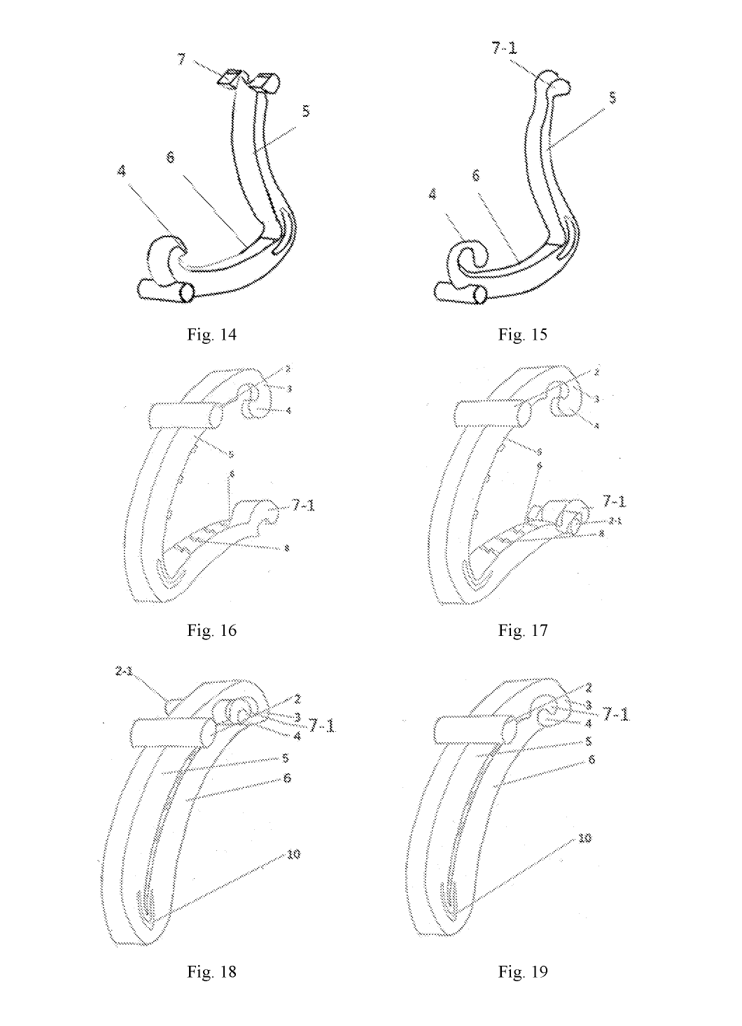

FIG. 14 is a graphic model showing the existing single tissue clamp with a bulge structure. FIGS. 1-4 & 7-9 show the paralleled structure of single tissue clamps based on existing techniques. FIGS. 5-6 show the duplex or double-head tissue clamp featuring the Tai Chi Double-Fish shaped hook-groove structure.

The hinge structure between the first curved clamping arm (5) and the second curved clamping arm (6) is described as follows: the joint between the first curved clamping arm and the second curved clamping arm is formed by two bifurcate connectors, i.e. an inner connector and an outer connector; and between the two connectors there is a hole (10). When the first curved clamping arm and the second curved clamping arm together form a V-shaped clamping body, the hole (10) is U-shaped. And when the clamping hook and groove at the free ends of the first and second curved clamping arms are engaged with each other, the tissue clamp is closed to form a narrow gap thus stopping flood flow. The stress resulting from the arc-shaped structure of the first and second curved clamping arms makes the hook and groove engage tightly. Whereas the pressure exerted on a biological tissue may not cause it to die or to operate, usually there is a narrow gap left between the closed clamping arms. And the clamping surfaces of the arms feature several tooth-shaped zones (8), which help generating a relatively larger friction between the biological tissue and the clamping arms. Through the design of the duplex tissue clamp (i.e. the double-head tissue clamp), the bumps can be located on the inner sides rather than outer sides. The connecting rods are between the paralleled clamps. The first bump acts as the third connecting rod (2) tying the two paralleled tissue clamps together, it can bulge and form the bump (9) on the outer sides of the clamping arms. The bump can also act as the supporting point for the clamp applier (however, this invention's duplex structure allows using the connecting rods as the supporting points for the applier). The design moves the bumps from outside to inside, so it is not so easy to injure blood vessels any more. Thanks to the ‘double-head/double-arm’ design and the additional connecting rod(s) between the two arms, the double-head tissue clamp has improved the clamp structure from two supporting points to three or four supporting points, thus enhancing the distortion resisting ability and making it possible to change the outer cross-section of the two arms from a square into an oval shape (i.e. the clamping arms can be designed with outer arcs). One or two connecting rods (11, 11-1) are located on the sides of the first or second curved clamping arm (it is allowable to adopt either connecting rods, or both connecting rods (but using the second connecting rod may influence the elasticity of the tissue clamp), or none of the connecting rods if the third and fourth connecting rods have already provided adequate fixation). The connecting rods can also act as the supporting points for the clamp applier. With reference to existing techniques, the third connecting rod (2) can bulge out and work together with the fourth connecting rod (which can also bulge out) as a supporting point for the applier. The first curved clamping arm (5) and the second curved clamping arm (6) are integrated to form the V-shaped clamping body. The free ends of the said first and second curved clamping arms are a clamping hook (4) and a clamping groove (7), respectively.

FIGS. 1-4 & 7-9 show ordinary duplex tissue clamps (i.e. simply connecting HEM-O-LOK in a paralleled manner).
a. The bump on the first curved clamping arms (5) goes through the paralleled arms and bulges out on the outer sides of the paralleled arms;
b. All bumps on the second curved short clamping arms are in the form of ‘cylinder+water droplet.’ The small bumps near the hinge between the two clamping arms are cylindrical and located on the back sides of the long arm (assuming that the long arm is divided into an outer layer and an inner layer, the outer layer can be understood as the back side and correspondingly the inner layer can be understood as the belly side);
c. If viewed from above, the width of a single arm of a medium-/large-sized duplex tissue clamp is 1 mm. The width of two arms is 1 mm+1 mm=2 mm. The spacing between two paralleled arms is 1 mm wide (so the width of a bump is also 1 mm). The head and tail of a bump on the outer sides of the duplex long arms bulge out by 1 mm, respectively; and those of a bump on the outer sides of the duplex short arms bulge out also by 1 mm, respectively. Therefore, the duplex tissue clamp is 5 mm wide in total. The spacing between the two paralleled arms of a small-sized duplex tissue clamp is 2 mm, so the small-sized duplex tissue clamp is 4 mm wide in total. Compared with the case of using two separate traditional tissue clamps simultaneously (two large-/medium-sized clamps require at least >6.5-7 mm, and two small-sized clamps require at least >4.5-5 mm), this invention has significantly saved the length of remnant blood vessel required in surgery, lowered the difficulty of clamping, simplified surgical operations, and improved surgeons' efficiency of single-pass clamping (the applying of two single tissue clamps, which traditionally requires operating twice, can now be done once only). As a surgical operation usually involves many clamping operations, the time required for completing a surgery can be reduced significantly through this invention.

FIG. 10 is a schematic diagram showing the section structure of the clamping arm and tooth. FIGS. 11a and 11b are schematic diagrams showing the layout of the tooth zones on the opened clamping arms of the duplex or double-head tissue clamp; i.e. schematic diagrams showing the layout of the two types of teeth on the clamping arms. The clamping surfaces of the clamping arms feature several tooth-shaped zones. In their free status, the first and second curved clamping arms form the V-shaped clamping body. The clamping surfaces of the arms have several tooth-shaped zones (8). Each line of oblique teeth is divided into an upper segment and a lower segment, of which the oblique teeth are arranged in a straight or staggered manner.

FIGS. 7-9 are schematic diagrams showing the section structure of the clamping hooks and grooves of the invented duplex or double-head tissue clamp, which is about to be closed (these diagrams correspond to the circumstances of adopting one upward bulge, two upward bulges and one/two upward bulges with a processed profile (7-2), respectively, at the joint of the hooks). The two tissue clamps in the figures have two clamping grooves corresponding to the clamping hooks. The clamping grooves are formed by the upward bulges on the sides of the second curved clamping arm. And the upward bulges between the two grooves are connected to form the fourth connecting rod tying the two tissue clamps together. The processed profile (7-2), i.e. the dent between the two upward bulges in the middle of the two clamping grooves, acts as a supporting end (force-applying point) for the clamp applier. Between the first and second curved clamping arms there is a ‘hinge’ structure. The clamping surfaces of the clamping arms feature several tooth-shaped zones. In their free status, the first and second curved clamping arms form the V-shaped clamping body. The first curved clamping arm (5) and second curved clamping arm (6) have one or two connecting rods (11) on their sides, tying the two paralleled tissue clamps together. The clamping hook (4) is connected with the first curved clamping arm (5) by an arc. Bump (2-2).

This invention mainly adopts biomedical polymer materials, such as ABS resin, polyether ether ketone (PEEK), reinforced polytetrafluoroethylene (reinforced PTFE), including UHMWPE, polypropylene, polypropylene or nylon. It can also adopt biocompatible materials, such as chitinous substances, as well as metallic or composite materials but with different manufacturing techniques. All these materials apply to the same structure design.

FIGS. 5-6 show the duplex or double-head tissue clamp featuring the Tai Chi Double-Fish shaped hook-groove structure. FIGS. 12-21 (excluding FIG. 14) show the Tai Chi Double-Fish shaped hook-groove structure. The first curved clamping arm (5) and the second curved clamping arm (6) are integrated to form the V-shaped clamping body. The free ends of the said first and second curved clamping arms are a clamping hook (4) and clamping groove (7-1), respectively. And the hook (4) and fish-shaped groove (7-1) also act as the groove and hook, respectively, for each other.

FIG. 12 provides two schematic diagrams showing the section structure of the engaged clamping hook and fish-shaped clamping groove of the invented clamp looks like a double-fish circle (FIG. 12a) or an oval variant (FIG. 12b). Such hook and groove can be lengthened or shortened, thus making the engagement of the hook and groove tighter or looser correspondingly, i.e. making it easier or harder to engage with each other. In fact, the clamping hook (4) and fish-shaped clamping groove (7-1) also work as the groove and hook, respectively, for each other. The clamping hook and fish-shaped clamping groove forms a double-fish circle if engaged with each other. However, there is a connecting section for each of the fish-shaped parts to link to the clamping arm, so there is a connecting section bulging around the circle structure.

FIG. 13 is a schematic diagram showing the section structure of the Tai Chi Double-Fish shaped hook and groove of the invented duplex or double-head tissue clamp, which is about to be closed. The third connecting rod (2-1) is located between the double-fish-shaped clamping grooves (7-1). When the duplex tissue clamp's double hooks (referring to the protruding parts) (4) engage in position, the third connecting rod (2-1) works as the guide block between the double hooks of the duplex tissue clamp.

FIG. 22 shows the form of the engaged clamping hook and groove of the invented duplex tissue clamp, i.e. a perspective side view of the double-fish-shaped three-head tissue clamp, with the forefront end of the third guide block designed as a hook to grasp the connecting rod (the hooking column) between the two hooks of the duplex tissue clamp; and the Tai Chi Double-Fish shaped structure can also be adopted for fitting the hook in the front of the third connecting rod onto the hooking column: this will constitute a double-fish-shaped three-head tissue clamp, with a big bump between the V-shaped paralleled curved arms (5 & 6) (this bump is transformed into a structure of ‘semicircle +fish-shaped hook opposite to the fish-shaped hooks on the two arms’); and there is an opening for the small bump near the hinge of the paralleled curved arms.

Compared with the double-fish-shaped double-head tissue clamp, the double-fish-shaped three-head tissue clamp offers one more clasping mechanism for the sake of security, i.e. adding a bump at the connecting part of the long arms' clamping hooks and designing such bump as a fish-shaped clasping hook, which makes the locking and clasping even more secure.

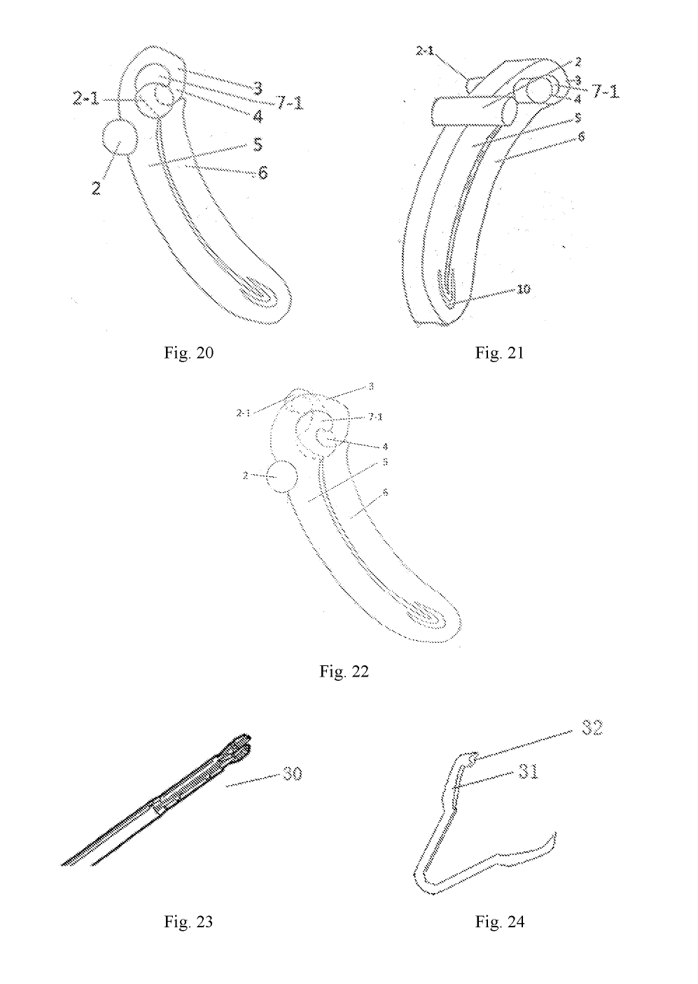

FIGS. 15-17 are schematic diagrams showing the Tai Chi double-fish-shaped hook-groove structure of several single tissue clamps set forth by this invention. All of them adopt similarly the Tai Chi Double-Fish shaped hook-groove structure, with small changes in their respective shape. And their shapes can be adjusted somehow to adapt to different requirements of material strength and toughness, etc. FIG. 18 is a schematic diagram showing the engaged status of the Tai Chi Double-Fish shaped hook and groove of the invented clamp (corresponding to FIG. 17). FIG. 19 is a schematic diagram showing the engaged status of the Tai Chi Double-Fish shaped hook and groove of the invented clamp (corresponding to FIG. 16). FIG. 20 is a schematic diagram showing the engaged status of the Tai Chi Double-Fish shaped hook and groove of the invented clamp (corresponding to FIG. 17). FIG. 21 is a schematic diagram showing the engaged status of the Tai Chi Double-Fish shaped hook and groove of the invented clamp (corresponding to FIG. 17). For single-hook structure, the fish-shaped clamping groove can be designed with upward bulges on both sides, i.e. the auxiliary guide block (2-1) so that the fish-shaped clamping hook would not go slipping when the clamp applier is applying a force onto it.

There is no bump between the V-shaped duplex clamping arms (5) and duplex clamping arms (6) (any bump between two arms can transform into a connecting rod/supporting rod), thus reducing the number of bumps (i.e. connecting rods/supporting rods) between the arms.

There are a big bump between the V-shaped duplex clamping arms (5) and duplex clamping arms (6) and a small bump near the hinge of the duplex clamping arms (5) and duplex clamping arms (6). A small bump can also be added near the hinge on the inner sides of the duplex short arms. The 3 (4) bumps ensure that the clamping body is sterically stabilized and not easy to deform in the course of clamping. For all bumps, A and B refer to supporting rods. The medium-/large-sized double-fish-shaped double-head tissue clamps not only change the clamping method of existing tissue clamps (changing the applier's holding positions from outer sides to inner sides) but also eliminate the bumps on the outer sides of the paralleled arms in design, achieving a total width of <5 mm, viewed from above, or a total width of <4 mm for the small-sized double-fish-shaped double-head tissue clamp.

The metallic jaws on the head of the clamp applier are aligned with the third and fourth connecting rods, which act as the force applying points. The part (31) is a metallic clamp handle.

This shall be understood by all technicians in relevant sectors: the abovementioned are only some specific examples of implementation of this invention, which are not meant to limit the scope of this invention; any and all changes, equivalent substitutions and improvements, etc., within the range of the spirit and principle of this invention shall be covered by the scope of protection of this invention.

1. A duplex tissue clamp, characterized in that two single tissue clamps connected in a parallel manner, wherein each of said paralleled tissue clamps includes a V-shaped clamping body formed by a first curved clamping arm and second curved clamping arm; free ends of said first and second curved clamping arms are a clamping hook and a clamping groove, respectively;
between the first and second curved clamping arms there is a ‘hinge’ structure, and in their free status, the first and second curved clamping arms form the V-shaped clamping body;
a first bump is located near a hook on adjacent inner sides of the first curved clamping arms, and the first bump connects the adjacent sides to form a third connecting rod tying the two paralleled tissue clamps together;
the two paralleled tissue clamps have two clamping grooves corresponding to the hooks;
an upward bulge is placed on the adjacent inner sides between the two clamping grooves, and the bulge acts as a fourth connecting rod tying the two paralleled tissue clamps together and the third and fourth connecting rods work as supporting points for a duplex tissue clamp applier.

2. The duplex tissue clamp according to claim 1 is characterized in that two upward bulges on adjacent sides between the two clamping grooves are merged into one middle upward bulge (as the fourth connecting rod and only one upward bulge is used to guide the two clamping hooks simultaneously.

3. The duplex tissue clamp according to claim 1 is characterized in that
the first connecting rod and/or the second connecting rod are located near the hinge and on the adjacent inner sides of the first and second curved clamping arms.

4. The duplex tissue clamp according to claim 1 is characterized in that when the two paralleled tissue clamps have an interval greater than 1.5-3 mm, a middle of the upward bulge is a widened structure and a supporting groove is disposed in the middle of the upward bulge to support the clamp applier.

5. The duplex tissue clamp according to claim 1 is characterized in that
the curved clamping arms of a medium or large-sized duplex tissue clamp are 2*1 mm in width an interval between the third and fourth connecting rods is 2 mm, and a total width of the two tissue clamp and the interval is 4 mm.

6. The duplex tissue clamp according to claim 1 is characterized in that
the first to fourth connecting rods of the two paralleled tissue clamps bulge on the outer sides of the two paralleled tissue clamps and constitute a bump, providing force-applying points for the clamp applier; and the third and fourth connecting rods can extend in the linear direction from the outer profile of the curved clamping arms to become a second bump, functioning as another force-applying point for the clamp applier.

7. The duplex tissue clamp according to claim 1 is characterized in that
clamping surfaces of the clamping arms are with several splay-styled anti-skid teeth, the teeth in anti-skid tooth-shaped zones of each of the paralleled tissue clamps resemble oblique lines; and in closed condition, the anti-skid tooth-shaped zones of the two tissue clamps resemble splayed lines each line of the teeth is divided into an upper segment and a lower segment, of which the teeth are arranged in a straight or staggered manner.

8. The duplex tissue clamp according to claim 7 is characterized in that
the clamping hook and fish-shaped clamping groove can fit into each other and form double-fish-shaped hooks, resembling a Tai Chi Double-Fish (Yin Yang Fish) diagram.

9. The duplex tissue clamp of claim 7, wherein the clamp applier for the duplex tissue clamp is characterized in that
when the two hooks of the double-head tissue clamp of the clamp applier are engaged with the fish-shaped clamping grooves, the third connecting rod functions as a guide block between the two hooks of a double-head tissue clamp; or
a forefront end of the guide block of the third connecting rod is made into a hook to catch on the connecting rod between the two hooks of the double-head tissue clamp.

10. The clamp applier designed for the duplex tissue clamp according to claim 9 is characterized in that
metallic jaws on the head of the clamp applier are aligned with the third and fourth connecting rods, which act as force applying points.
